(19) United States
(12) Patent Application Publication
(10) Pub. No.: US 2017/0097355 A1
(43) Pub. Date: Apr. 6, 2017

(54) BIOMARKERS AND METHODS TO DISTINGUISH OVARIAN CANCER FROM BENIGN TUMORS

(71) Applicant: UNIVERSITY OF WASHINGTON, Seattle, WA (US)

(72) Inventors: Daniel RAFTERY, SEATTLE, WA (US); Haiwei GU, SEATTLE, WA (US); Chris LI, SEATTLE, WA (US)

(73) Assignee: UNIVERSITY OF WASHINGTON, SEATTLE, WA (US)

(21) Appl. No.: 15/287,511

(22) Filed: Oct. 6, 2016

Related U.S. Application Data
(60) Provisional application No. 62/238,036, filed on Oct. 6, 2015.

(51) Int. Cl. G01N 33/574

(52) U.S. Cl. CPC G01N 33/57449; G01N 33/57484

(57) ABSTRACT

Methods for detecting and measuring metabolic changes useful in the detection of cancer, and in differentiating between ovarian cancer and benign ovarian tumor are described, as well as the unexpected and valuable combination of detecting both lipidomic and aqueous metabolites. Two independent LC-MS-based metabolomics platforms, including a global lipidomics approach, were used to screen for differentially abundant plasma metabolites between cases with serous ovarian carcinoma and controls with benign serous ovarian tumor. The identified biomarkers can be used to distinguish between ovarian cancer and benign tumors. Standards and kits for use with the methods for detecting cancer are also provided.

This application claims benefit of U.S. provisional patent application No. 62/238,036, flied Oct. 6, 2015, the entire contents of which are incorporated by reference into this application.

### ACKNOWLEDGEMENT OF GOVERNMENT SUPPORT

This invention was made with government support under Grant No. U01 CA152637-04S1, awarded by the National Institutes of Health. The government has certain rights in the invention.

### TECHNICAL FIELD OF THE INVENTION

The present invention relates to biomarkers and their use for detecting cancer and for distinguishing serous ovarian carcinoma and benign serous ovarian tumors, as well as kits, standards, and methods for measuring the biomarkers in samples obtained from subjects.

### BACKGROUND OF THE INVENTION

Serous ovarian carcinoma (OC) represents a leading cause of cancer-related death among U.S. women [1]. Among women who present with an ovarian mass and are referred for surgical excision, an important clinical challenge involves identifying, prior to surgery, those with malignancy versus benign disease. Prognosis and survival are significantly improved when surgical interventions are performed by tertiary care specialists [2, 3] Recently-developed methods for assessing the likelihood of cancer, such as the Risk of Ovarian Malignancy Algorithm (ROMA) and the “OVA1” test, are based on blood levels of CA125 and other proteins-HE4, prealbumin, transferrin, β2 microglobulin, and apolipoprotein Al [4-8]. These approaches have shown promise but continue to be evaluated, and will likely require further validation in multi-center trials prior to widespread clinical adoption [9-14].

Metabolomics has emerged as a promising domain for identifying molecular signatures associated with cancer [15-17], In contrast to genomics, transcriptomics, or proteomics, metabolomics describes the study of concentrations and fluxes of low molecular weight metabolites in biofluids or tissue [18, 19]. As metabolic fluctuations he downstream of alterations at the DNA, RNA, and protein levels, metabolomics offers a sensitive and comprehensive functional read-out of biological systems. Metabolite alterations in tissue or biofluids have been linked to a broad spectrum of human cancers [20-24]. Adaptive changes such as increased rates of aerobic glycolysis (the Warburg effect), amino acid metabolism, and lipid turnover are thought to be important features of the neoplastic process that facilitate sustained cellular proliferation and tumor expansion [15].

Several past metabolomics studies have focused on OC, and used liquid chromatography (LC)-mass spectrometry (MS), gas chromatography (GC)-MS, or nuclear magnetic resonance (NMR) to evaluate tissue [25, 26], blood [27-30], or urine [31, 32]. While these reports have described changes in the levels of multiple classes of metabolites (purines/pyrimidines, lipids, tricarboxylic acid (TCA) intermediates, and amino acids) in women with versus without ovarian cancer, minimal consensus has emerged with respect to robust, consistent metabolic signatures, either within or across biospecimen types (tissue, blood, urine). Nevertheless, three independent studies have implicated alterations in lipids and/or phospholipids in either plasma or tissue from OC cases [25, 29, 30], Comprehensive lipidomics profiling on OC specimens, however, has not been conducted previously.

There remains a need to identify useful biomarkers for the detection and monitoring of ovarian cancer, and for methods of measuring such biomarkers.

### SUMMARY OF THE INVENTION

In one embodiment, the invention provides a method of determining the amounts of metabolites in a sample. The method comprises measuring the concentrations of at least two components of a panel of a plurality of metabolites in a sample obtained from a subject, and determining a ratio of the concentration of each of the components to control concentration of each of the components. The components of the panel are selected from the group consisting of: C52 H79 N O5 G. PS(O-18:0/0:0), 18:3 Cholesteryl ester+22.7, TG(16:0/16:1(9Z)/16:1 (9Z))[iso3]+23,5, PG(P-20:0/12:0), TG(16:1(9Z)/16:1(9Z)/16:1(9Z)) +227, PS(O-18:0/16: 1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)117:1(9Z)117:2(9Z,12Z)) [iso6], PE(18:1(9420:3(8Z,11Z,14Z)), C57 H102 N2 O S, TG(16:0116:0116:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z)) [iso6], and Alanine.

In some embodiments, the method further comprises detecting OC in the subject when the determined ratio is less than 1 for each of the components, In one embodiment, the at least two components comprises Alanine. In another embodiment, the at least two components comprises C52 H79 N O5 and PS(O-18:0/0:0). In some embodiments, the measuring comprises measuring the concentrations of at least 3 components of the panel. In one embodiment, the at least 3 components of the panel comprises C52 H79 N O5 S, PS(O-18:0/0:0), and Alanine. In other embodiments, the measuring comprises measuring the concentrations of at least 4 components of the panel. In one such embodiment, the at least 4 components of the panel comprises C52 H79 N O5 S, PS(O-18:0/0:0), 18:3 Cholesteryl ester+22.7, and TG(16:0/16:1(9Z)/16:1(9Z))[iso3]+23.5. In other embodiments, the measuring comprises measuring the concentrations of at least 5 components of the panel. In one such embodiment, the at least 5 components of the panel comprises C52 H79 N O5 S, PS(O-18:0/0:0), 18:3 Cholesteryl ester+22.7, TG(16:0/16:1(9Z)/16:1(9Z)[iso3]+23.5, and Alanine,

In one embodiment, the measuring comprises measuring the concentrations of at least 17 components of the panel. For example, the at least 17 components of the panel can comprise: C52 H79 N O5 S, PS(O-18:0/0:0), 18:3 Cholesteryl ester+22.7, TG(16:0/16:1(9Z)/16:1(9Z))[iso3]+23.5, PG(P-20:0/12:0), TG(16:1(9Z)/16:1(9Z)/16:1(9Z))+22.7, PS(O-18:0/16:1(9Z)), TG(17:2(9Z,12Z)/17:2(9Z, 12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)117:1(9Z)117:2(9Z,12Z)) [iso6], PE(18:1(9Z)/20:3(8Z,11Z,14Z)), C57 H102 N2 O S, TG(16:0/16:0/16:1(9Z)[iso3],C29 H47 N9 O2, PS(O-20:0/0:0), PE(18:1(9Z)/20:3(8Z,11Z,14Z)), and TG(16:1(9Z)117:0/17:2 (9Z,12Z))[iso6]. In another embodiment, the method comprises measuring the concentrations of at least 18 components of a panel of a plurality of metabolites in a sample from the subject. The at least 18 components of the panel comprises C52 H79 N O5 S, PS(O-18:0/0:0), 18:3 Cholesteryl ester+22.7, TG(16:0/16:1(9Z))[iso3]+23.5, PG(P-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 H9 O2, PS(O-20:0/0:0), PE(18:1(9Z)/20:3(8Z,11Z,14Z)), TG(16:1(9Z)/17:0/17:2(9Z,12Z))[iso6], and Alanine.

In some embodiments, the method further comprises measuring CA125 and/or HE4 in a sample from the subject, wherein a statistically significant increase in CA125 and/or HE4 relative to a control sample is indicative of OC. In some embodiments, the method further comprises measuring prealbumin and/or transferrin in a sample from the subject, wherein a statistically significant decrease in prealbumin and/or transferrin relative to a control sample is indicative of OC.

In one embodiment, the sample is obtained from a subject suspected of having ovarian cancer (OC). In another embodiment, the sample is obtained from a subject presenting with a pelvic and/or ovarian mass or tumor. In some embodiments, the control sample is obtained from a normal, healthy subject or obtained from the subject at an earlier time.

Also described herein is a method of detecting ovarian cancer in a subject, and a method of screening for ovarian cancer. The invention additionally provides a method of distinguishing between benign and malignant tumors. In one embodiment, the tumors are ovarian tumors. The invention further provides a method of treating ovarian cancer in a subject. The method comprises measuring the concentrations of at least two components of a panel of a plurality of metabolites in a sample obtained from a subject, determining a ratio of the concentration of each of the components to control concentration of each of the components, and treating the subject for ovarian cancer when the determined ratio is less than 1 for each of the components.

In some embodiments, the measuring comprises liquid chromatography, mass spectrometry, enzymatic assay, and/or immunoassay. In some embodiments, the liquid chromatography may comprise high performance (HPLC), ultra performance (UPLC), turbulent flow (TFLC), or any combination thereof. In some embodiments, at least one purification step and mass spectrometric analysis is conducted in an on-line fashion. In another embodiment, the mass spectrometry is tandem mass spectrometry (MS/MS) or quadrupole time of flight (QTOF) mass spectrometry.

In certain preferred embodiments of the methods disclosed herein, mass spectrometry is performed in positive ion mode. Alternatively, mass spectrometry is performed in negative on mode. Various ionization sources, including for example atmospheric pressure chemical ionization (APCI) or electrospray ionization (ESI), may be used in embodiments of the present invention.

In some embodiments, one or more separately detectable standards is provided in the sample, the amount of which is also determined in the sample. An internal standard may be used to account for loss of analytes during sample processing in order to get a more accurate value of a measured metabolite in the sample. In these embodiments, all or a portion of one or more components selected from the group consisting of the panel of a plurality of metabolites, and the one or more standards present in the sample are ionized to produce a plurality of ions detectable in a mass spectrometer. In preferred embodiments, the amount of ions generated from a component of interest may be related to the presence of amount of component of interest in the sample by comparison to one or more internal standards.

The invention further provides a kit comprising a set of standards. The set comprises at least three standards of the following: C52 H79 N O5 S, PS(O-18:0/0:0), 18:3 Cholesteryl ester+22.7, TG(16:0/16:1(9Z)/16:1(9.Z))[iso3]+23.5, PG(O-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z))[iso6], In some embodiments, the kit further comprises reagents for detection of alanine.

For use in the methods described herein, representative examples of the sample include, but are not limited to, blood, plasma or serum, saliva, urine, cerebral spinal fluid, milk, cervical secretions, semen, tissue, cell cultures, and the like. In one embodiment, the sample is plasma.

### BRIEF DESCRIPTION OF THE DRAWINGS

FIG. 1. Box plots for selected top differentiating metabolites between benign controls (white) and OC cases (gray). Covariate-adjusted metabolite values were plotted by case status, stratified by training or testing set. Median metabolite signals are indicated by solid black lines. Values for the 25th percentile and 75th percentile are indicated by the lower and upper box boundaries, respectively, with dotted lines marking the distribution of observed values, and outliers indicated by gray dots.

FIG. 2. Receiver operating characteristic (ROC) curves for multivariate classifiers based on the top four lipid metabolites with or without CA125, and for CA125 alone. Study participants (n=84) were randomly divided into a training (75%) or testing (25%) set. A multivariate classifier was derived in the training set using the top four lipid metabolites (Table 2A), and evaluated in the testing set. After 100 rounds of Monte Carlo cross validation (MCCV), a composite average ROC curve was generated, The procedure was repeated using these four lipid metabolites plus CA125, and performance of CA125 alone was similarly assessed via the MCCV framework: Mean area under the ROC curve (AUC) was calculated across all MCCV iterations. Estimated specificity at 95% sensitivity indicated by black circles.

FIG. 3. Receiver operating characteristic (ROC) curves for multivariate classifiers based on the top 17 lipid metabolites with or without CA125, and for CA125 alone. Study participants (n=84) were randomly divided into a training (75%) or testing (25%) set. A multivariate classifier was derived in the training set using the top 17 lipid metabolites (Table 2A: FDR q<0.05), and evaluated in the testing set. After 100 rounds of Monte Carlo cross validation (MCCV), a composite average ROC curve was generated: The procedure was repeated using these 17 lipid metabolites plus CA125, and performance of CA125 alone was similarly assessed via the MCCV framework. Mean area under the ROC curve (AUC) was calculated across all MCCV iterations, and specificity at 95% sensitivity was estimated as indicated (black circles).

FIG. 4. Performance comparison of models constructed with CA125 and either the top 17 lipid metabolites (FIG. 3) or the top four lipid metabolites (FIG. 2): Horizontal dashed line near top corresponds to 95% sensitivity.

### DETAILED DESCRIPTION OF THE INVENTION

The invention provides new methods for detecting and measuring metabolic changes useful in the detection of cancer, and in differentiating between ovarian cancer and benign ovarian tumor. Also described herein is the unexpected and valuable combination of detecting both lipidomic and aqueous metabolites, As described in the Examples below, two independent LC-MS-based metabolomics platforms, including a recently-optimized in-house global lipidomics approach, were used to screen for differentially abundant plasma metabolites between cases with serous ovarian carcinoma and controls with benign serous ovarian tumor. Plasma samples isolated at the time of surgery from 50 cases and 50 controls were selected from a high-quality local biorespository and subjected to i) global lipidomics profiling of >600 lipid metabolites, and ii) targeted aqueous profiling [24] of ˜160 selected metabolites encompassing several major metabolic pathways. The potential utility of derived metabolite profiles to discriminate between cases and controls was investigated through construction and evaluation of multivariate classification models.

### Definitions

All scientific and technical terms used in this application have meanings commonly used in the art unless otherwise specified. As used in this application, the following words or phrases have the meanings specified.

As used herein, an “isotopic label” produces a mass shift in the labeled molecule relative to the unlabeled molecule when analyzed by mass spectrometric techniques. Examples of suitable labels include deuterium, ¹³C, and ¹⁵N. Deuterium is a useful label because it can potentially produce three mass unit shifts in a labeled methylation product relative to an unlabeled methylation product. An isotopic label may be incorporated at one or more positions in the molecule and one or more kinds of isotopic labels may be used on the same isotopically labeled molecule.

As used herein, the term “purification” or “purifying” refers to enriching the amount of one or more components of interest relative to other components in the sample that may interfere with detection of the metabolite of interest. Purification of the sample leads to relative reduction of one or more interfering substances, e.g., one or more substances that may or may not interfere with the detection of selected parent or daughter ions by mass spectrometry. Relative reduction as this term is used does not require that any substance, present with the analyte of interest in the material to be purified, is entirely removed by purification.

As used herein, the term “sample” refers to any sample that may contain an analyte of interest. As used herein, the term “body fluid” means any fluid that can be isolated from the body of an individual. For example, “body fluid” may include blood, plasma, serum, bile, saliva, urine, tears, perspiration, and the like. Typical samples for use in the present invention comprise human serum or plasma.

As used herein, the term “solid phase extraction” or “SPE” refers to a process in which a chemical mixture is separated into components as a result of the affinity of components dissolved or suspended in a solution (i.e., mobile phase) for a solid through or around which the solution is passed (i.e., solid phase). In some instances, as the mobile phase passes through or around the solid phase, undesired components of the mobile phase may be retained by the solid phase resulting in a purification of the analyte in the mobile phase. In other instances, the analyte may be retained by the solid phase, allowing undesired components of the mobile phase to pass through or around the solid phase. In these instances, a second mobile phase is then used to elute the retained analyte off of the solid phase for further processing or analysis.

As used herein, the term “chromatography” refers to a process in which a chemical mixture carried by a liquid or gas is separated into components as a result of differential distribution of the chemical entities as they flow around or over a stationary liquid or solid phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

As used herein, the term “high performance liquid chromatography” or “HPLC” (also sometimes known as “high pressure liquid chromatography”) refers to liquid chromatography in which the degree of separation is increased by forcing the mobile phase under pressure through a stationary phase, typically a densely packed column. As used herein, the term “ultra high performance liquid chromatography” or “UPLC” or “UHPLC” (sometimes known as “ultra high pressure liquid chromatography”) refers to HPLC which occurs at much higher pressures than traditional HPLC techniques.

As used herein, the term “on-line” or “inline,” for example as used in “on-line automated fashion” or “on-line extraction,” refers to a procedure performed without the need for operator intervention. In contrast, the term “off-line” as used herein refers to a procedure requiring manual intervention of an operator. Thus, if samples are subjected to precipitation, and the supernatants are then manually loaded into an autosampler, the precipitation and loading steps are off-line from the subsequent steps. In various embodiments of the methods, one or more steps may be performed in an on-line automated fashion.

As used herein, the term “sample injection” refers to introducing an aliquot of a single sample into an analytical instrument, for example a mass spectrometer. This introduction may occur directly or indirectly. An indirect sample injection may be accomplished, for example, by injecting an aliquot of a sample into a HPLC or UPLC analytical column that is connected to a mass spectrometer in an on-line fashion.

As used herein, the term “same sample injection” with respect to multiple analyte analysis by mass spectrometry means that the ions for two or more different analytes are determined essentially simultaneously by measuring ions for the different analytes from the same (i.e. identical) sample injection.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass ('m”) and charge (“z”), See, e.g., U.S. Pat. No. 6,204,500, entitled “Mass Spectrometry From Surfaces;” U.S. Pat. No. 6,107,623, entitled “Methods and Apparatus for Tandem Mass Spectrometry:” U.S. Pat. No, 6,268,144, entitled “DNA Diagnostics Based On Mass Spectrometry” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2; 264-76; and Merchant and Weinberger, Electrophoresis 2000, 21; 1164-67.

As used herein, the term “operating in negative ion mode” refers to those mass spectrometry methods where negative ions are generated and detected, The term “operating in positive ion mode” as used herein, refers to those mass spectrometry methods where positive ions are generated and detected.

As used herein, the term “ionization” or “ionizing” refers to the process of generating an analyte ion having a net electrical charge equal to one or more electron units, Negative ions are those having a net negative charge of one or more electron units, while positive ions are those having a net positive charge of one or more electron units.

As used herein, the term “electron ionization” or “EI” refers to methods in which an analyte of interest in a gaseous or vapor phase interacts with a flow of electrons. Impact of the electrons with the analyte produces analyte ions, which may then be subjected to a mass spectrometry technique,

As used herein, the term “chemical ionization” or “CI” refers to methods in which a reagent gas (e.g. ammonia) is subjected to electron impact, and analyte ions are formed by the interaction of reagent gas ions and analyte molecules.

As used herein, the term “fast atom bombardment” or “FAB” refers to methods in which a beam of high energy atoms (often Xe or Ar) impacts a non-volatile sample, desorbing and ionizing molecules contained in the sample. Test samples are dissolved in a viscous liquid matrix such as glycerol, thioglycerol, m-nitrobenzyl alcohol, 18-crown-6 crown ether, 2-nitrophenyloctyl ether, sulfolane, diethanolamine, and triethanolamine. The choice of an appropriate matrix for a compound or sample is an empirical process.

As used herein, the term “matrix-assisted laser desorption ionization” or “MALDI” refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

As used herein, the term “surface enhanced laser desorption ionization” or “SELDI” refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

As used herein, the term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

As used herein, the term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N.sub.2 gas may be used to improve removal of solvent. The gas-phase ionization in APCI can be more effective than ESI for analyzing less-polar species.

The term “atmospheric pressure photoionization” or “APPI” as used herein refers to the form of mass spectrometry where the mechanism for the photoionization of molecule M is photon absorption and electron ejection to form the molecular ion M+. Because the photon energy typically is just above the ionization potential, the molecular on is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Drug compounds in protic solvents are usually observed as MH+, whereas nonpolar compounds such as naphthalene or testosterone usually form M+. See, e.g., Robb et al., Anal. Chem. 2000, 72(15): 3653-3659.

As used herein, the term “inductively coupled plasma” or “ICP” refers to methods in which a sample interacts with a partially ionized gas at a sufficiently high temperature such that most elements are atomized and ionized.

As used herein, the term “field desorption” refers to methods in which a non-volatile test sample is placed on an ionization surface, and an intense electric field is used to generate analyte ions.

As used herein, the term “desorption” refers to the removal of an analyte from a surface and/or the entry of an analyte into a gaseous phase. Laser desorption thermal desorption is a technique wherein a sample containing the analyte is thermally desorbed into the gas phase by a laser pulse. The laser hits the back of a specially made 96-well plate with a metal base. The laser pulse heats the base and the heats causes the sample to transfer into the gas phase. The gas phase sample is then drawn into the mass spectrometer.

As used herein, the term “selective ion monitoring” is a detect on mode for a mass spectrometric instrument in which only ions within a relatively narrow mass range, typically about one mass unit, are detected.

As used herein, “multiple reaction mode,” sometimes known as “selected reaction monitoring,” is a detection mode for a mass spectrometric instrument in which a precursor ion and one or more fragment ions are selectively detected.

As used herein, an “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

The term “about” as used herein in reference to quantitative measurements not including the measurement of the mass of an ion, refers to the indicated value plus or minus 10%. Mass spectrometry instruments can vary slightly in determining the mass of a given analyte. The term “about” in the context of the mass of an ion or the mass/charge ratio of an ion refers to +/−0.50 atomic mass unit.

As used herein, “a” or “an” means at least one, unless clearly indicated otherwise.

### Methods of the Invention

The invention provides methods for detecting and monitoring cancer, including ovarian cancer, as well as of distinguishing between ovarian cancer (OC) and benign tumors, These methods comprise detecting and/or measuring the amounts of metabolites in a sample obtained from a subject. In one embodiment, the invention provides a method of determining the amounts of metabolites in a sample. The method comprises measuring the concentrations of at least two components of a panel of a plurality of metabolites in a sample obtained from a subject, and determining a ratio of the concentration of each of the components to control concentration of each of the components: The components of the panel are selected from the group consisting of: C52 H79 N O5 S, PS(O-18:0/0:0), 18:3 Cholesteryl ester+22.7, TG(16:0/16:1(9Z)/16:1 (9Z))[iso3]+23.5, PG(O-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z)[iso3]+22.2, TG(16:1(9Z)/17:1(9Z)/17:2(9Z,12Z)) [iso6], PE(18:1(9Z)/20:3(8Z,11Z,14Z)), C57 H102 N2 O S, TG(16:0/16:0/16:1(9Z))[iso3], C29 H47 N9 O2, P5(O-20:0/0:0), PE(18:1(9Z)/20:3(8Z,11Z,14Z)), TG(16:1(9Z)/17:0/17:2(9Z,12Z)) [iso6], and Alanine.

In some embodiments, the method further comprises detecting OC in the subject when the determined ratio is less than 1 for each of the components. In one embodiment, the at least two components comprise Alanine. In another embodiment, the at least two components comprises C52 H79 N O5 and PS(O-18:010:0). In some embodiments, the measuring comprises measuring the concentrations of at least 3 components of the panel. In one embodiment, the at least 3 components of the panel comprises C52 H79 N O5 S, PS(O-18:0/0:0), and Alanine. In other embodiments, the measuring comprises measuring the concentrations of at least 4 components of the panel. In one such embodiment, the at least 4 components of the panel comprises C52 H79 N O5 S, PS(O-18:0/0:0), 18:3 Cholesteryl ester+22.7, and TG(16:0/16:1(9Z)/16:1(9Z))[iso3]+23.5. In other embodiments, the measuring comprises measuring the concentrations of at least 5 components of the panel. In one such embodiment, the at least 5 components of the panel comprises C52 H79 N O5 S, PS(O-18:0/0:0), 18:3 Cholesteryl ester+22.7, TG(16:0/16:1(9Z)/16:1(9Z))[iso3]+23.5, and Alanine,

In one embodiment, the measuring comprises measuring the concentrations of at least 17 components of the panel. For example, the at least 17 components of the panel can comprise: C52 H79 N O5 S, PS(O-18:010:0), 18:3 Cholesteryl ester+22.7, TG(16:0/16:1(9Z)/16:1(9Z))[iso3]+23.5, PG(O-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z)) [iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), and TG(16:1(9Z)/17:0/17:2(9Z,12Z))[iso6]. In another embodiment, the method comprises measuring the concentrations of at least 18 components of a panel of a plurality of metabolites in a sample from the subject. The at least 18 components of the panel comprises C52 H79 N O5 S, PS(O-18:0/0:0), 18:3 Cholesteryl ester+22:7, TG(16:0/16:1(9Z)/16:1(9Z))[iso3]+23.5, PG(P-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z)[iso6], and Alanine.

In some embodiments, the method further comprise measuring CA125 and/or HE4 in a sample from the subject, wherein a statistically significant increase in CA125 and/or HE4 relative to a control sample is indicative of OC. In some embodiments, the method further comprises measuring prealbumin and/or transferrin in a sample from the subject, wherein a statistically significant decrease in prealbumin and/or transferrin relative to a control sample is indicative of OC.

In one embodiment, the sample is obtained from a subject suspected of having ovarian cancer (OC). In another embodiment, the sample is obtained from a subject presenting with a pelvic and/or ovarian mass. In some embodiments, the control sample is obtained from a normal, healthy subject or obtained from the subject at an earlier time.

Also described herein is a method of detecting ovarian cancer in a subject, and a method of screening for ovarian cancer. The invention additionally provides a method of distinguishing between benign and malignant tumors. In one embodiment, the tumors are ovarian tumors. The invention further provides a method of treating ovarian cancer in a subject. The method comprises measuring the concentrations of at least two components of a panel of a plurality of metabolites in a sample obtained from a subject, determining a ratio of the concentration of each of the components to control concentration of each of the components, and treating the subject for ovarian cancer when the determined ratio is less than 1 for each of the components.

For use in the methods described herein, representative examples of the sample include, but are not limited to, blood, plasma or serum, saliva, urine, cerebral spinal fluid, milk, cervical secretions, semen, tissue, cell cultures, and other bodily fluids or tissue specimens.

In some embodiments, the measuring comprises liquid chromatography, mass spectrometry, enzymatic assay, and/or immunoassay, in some embodiments, the liquid chromatography may comprise high performance (HPLC), ultra performance (UPLC), turbulent flow (TFLC), or any combination thereof: In some embodiments, at least one purification step and mass spectrometric analysis is conducted in an on-line fashion. In another embodiment, the mass spectrometry is tandem mass spectrometry (MS/MS) or quadrupole time of flight (QTOF) mass spectrometry.

In certain preferred embodiments of the methods disclosed herein, mass spectrometry is performed in positive ion mode. Alternatively, mass spectrometry is performed in negative ion mode. Various ionization sources, including for example atmospheric pressure chemical ionization (APCI) or electrospray ionization (ESI), may be used in embodiments of the present invention,

In some embodiments, one or more separately detectable standards is provided in the sample, the amount of which is also determined in the sample. An internal standard may be used to account for loss of analytes during sample processing in order to get a more accurate value of a measured metabolite in the sample. In these embodiments, all or a portion of one or more components selected from the group consisting of the panel of a plurality of metabolites, and the one or more standards present in the sample are ionized to produce a plurality of ions detectable in a mass spectrometer. In preferred embodiments, the amount of ions generated from a component of interest may be related to the presence of amount of component of interest in the sample by comparison to one or more internal standards.

### Kits

The invention provides kits comprising a set of standards. The set comprises at least three standards of the following: C52 H79 N O5 G. PS(O-18:0/0:0), 18:3 Cholesteryl ester+22:7, TG(16:0/16:1(9Z)/16:1(9Z))[iso3]+23.5, PG(P-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z)) [iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z)) [iso6]. In some embodiments, the kit further comprises reagents for detection of alanine.

Kits can include more than three standards, and in some embodiments, as many as 10, 15, 20, or more standards. The kit can optionally include a buffer, additional reagents, and other elements useful for the optimization of the measurement of select biomarkers. Optionally included are containers housing the standards, reagents, and other elements of the kit, as well as instructions for using same.

### EXAMPLES

The following examples are presented to illustrate the present invention and to assist one of ordinary skill in making and using the same. The examples are not intended in any way to otherwise limit the scope of the invention.

### Example 1: Identification of Novel Candidate Plasma Metabolite Biomarkers for Distinguishing Serous Ovarian Carcinoma and Benign Serous Ovarian Tumors

Using liquid chromatography-mass spectrometry, this Example describes global and targeted metabolite profiling of plasma isolated at the time of surgery from 50 serous OC cases and 50 serous benign controls. This global lipidomics analysis identified 34 metabolites (of 372 assessed) differing significantly (P<0.05) between cases and controls in both training and testing sets, with 17 candidates satisfying FDR q<0.05, and two reaching Bonferroni significance. Targeted profiling of ˜150 aqueous metabolites identified a single amino acid, alanine, as differentially abundant (P<0.05), A multivariate classification model built using the top four lipid metabolites achieved an estimated AUC of 0.85 (SD=0.07) based on Monte Carlo cross validation. Evaluation of a hybrid model incorporating both CA125 and lipid metabolites was suggestive of increased classification accuracy (AUC=0.91, SD=0.05) relative to CA125 alone (AUC=0.87. SD=0.07), particularly at high fixed levels of sensitivity, without reaching significance. These results provide insight into metabolic changes potentially correlated with the presence of OC versus benign ovarian tumor and demonstrate that plasma metabolites can be used to differentiate these two conditions.

### Subjects and Methods

Participants were recruited between May 2006 and August 2011 to support protocols of the Pacific Ovarian Cancer Research Consortium (POCRC) by physicians at Pacific Gynecology Specialists, Swedish Medical Center (Seattle, Wash.). Specimen collection was funded by the National Cancer Institute's Specialized Programs of Research Excellence (SPORE) program. AM subjects were post-menopausal women who had been referred for surgical excision of an ovarian mass, and had no prior history of cancer. 50 cases with serous ovarian carcinoma and 50 controls with benign serous ovarian tumor were selected for analysis. Controls were frequency-matched to cases by age, ethnicity, body-mass index (BMI), parity, use of oral contraceptives and year of blood draw. All diagnoses were made in accordance with uniform histological and pathological guidelines. Demographic and lifestyle characteristics were obtained through questionnaires filled out by patients prior to surgery. Cases and controls were randomly and equally distributed into either a training set (n=50) or testing set (n=50). This study was approved by the Institutional Review Board at the Fred Hutchinson Cancer Research Center.

### Plasma Specimens

Twelve-hour fasted blood samples from included study participants were collected at the time of surgery and processed according to standardized protocols, as described previously [33]. All blood specimens were processed within four hours post-draw. Plasma samples selected for analysis were distributed into three batches, such that case and control specimens were equally represented and randomly ordered within-batch. Each sample was assigned a unique laboratory identification number, which specified the order of processing and blinded laboratory personnel to sample identities. Specimens were delivered on dry ice to the Northwest Metabolomics Research Center and stored at −80° C. until use.

### Sample Preparation

Detailed protocols for the preparation of samples analyzed via targeted aqueous metabolite profiling have been published previously [24]. For global lipidomics, frozen plasma samples were thawed at room temperature for 30 min. 20 μL of each sample was mixed with 200 μL chloroform/methanol (2:1, v:v). The chloroform/methanol solution contains the following five lipids standards: PC(17:0/0:0) at 8.75 μM, PC(17:0/17:0) at 5.85 μM, PE (17:0/17:0) at 6.19 μM, TG(17:0/17:0/17:0) at 4.20 μM, and Cer(d18:1/17:0) at 6.46 μM. The lipid standards were used to monitor sample analysis performance, and were purchased from Avanti Polar Lipids, Inc (Alabaster, Alabama). The mixture was then vortexed for 2 minutes and incubated for 30 minutes at room temperature (25° C.), followed by centrifugation at 14,000×g for 5 min. 120 μL of the lower (organic) phase were then collected and loaded into a glass vial for lipidomics analysis.

### LC-MS/MS Analysis

Targeted LC-MS/MS experiments were performed on an Agilent 1260 LC (Agilent Technologies, Santa Clara, Calif.) AB Sciex QTrap 5500 MS (AB Sciex, Toronto, Canada) system, controlled by Analyst 1.5 software (AB Sciex, Toronto, Canada), as described previously [24]. Two sample injections (2 μL and 10 μL) were used for LC-MS/MS analysis in positive and negative ion modes, respectively. To assess instrument performance and process reproducibility, replicates of two independent quality control (QC) serum samples were injected after every 10 patient samples. Chromatographic separations were performed in hydrophilic interaction chromatography (HILIC) mode (SeQuant ZIC-cHILIC, 150×2.1 mm, 3.0 μm, Merck KGaA, Darmstadt, Germany). The flow rate and column temperature were set to 300 μL/min and 45° C., respectively. Multiple reaction-monitoring (MRM) mode was used to detect metabolites of interest. The 154 metabolites selected for targeted analysis represent numerous major metabolic pathways (eg. glycolysis/TCA cycle, amino acid, nucleotide, and lipid metabolism) [24].

### Global Lipidomics Analysis

LC-QTOF-MS experiments were performed using an Agilent 1200 SL LC system coupled online with an Agilent 6520 Q-TOF mass spectrometer (Agilent Technologies, Santa Clara, Calif.). Each prepared sample (4 μL for positive ESI ionization_{;} 8 μL for negative ESI ionization) was injected onto an Agilent Zorbax 300 SB-C8 column (2.1×50 mm, 1.8-micron), which was heated to 50° C. The flow rate was 0.4 mL/min. Mobile phase A was 5 mM ammonium acetate and 0.1% formic acid in water, and mobile phase B was 5% water in ACN containing 5 mM ammonium acetate and 0.1% formic acid. The mobile phase composition was kept isocratic at 35% B for 1 min, and was increased to 95% B in 19 min; after another 10 min at 95% B, the mobile phase composition was returned to 35% B. The ESI voltage was 3.8 kV. The mass accuracy of our LC-MS system is generally less than 5 ppm; the Q-TOF MS spectrometer was calibrated prior to each batch run, and a mass accuracy of less than 1 ppm was often achieved using the standard tuning mixture (G1969-85000, Agilent Technologies, Santa Clara, Calif.). The mass scan range is 100-1600, and the acquisition rate was 1.5 spectra/s. The absolute intensity threshold for MS data collection was set to 100, and the relative threshold was 0.001%.

### Measurement of CA125

A customized in-house bead-based ELISA assay was used to assess plasma CA125 levels, as described previously [33, 34] All measurements were conducted in the laboratory of Dr. Nicole Urban at the Fred Hutchinson Cancer Research Center.

### Data Processing and Regression Analyses

For global lipidomics profiling, raw spectra were processed using MassHunter Qualitative Analysis Software and Mass Profiler Professional Software (Agilent). For targeted aqueous profiling, peak intensities for detected metabolites were integrated using Analyst 1.5 software (AB SCIEX). Peak integrals were used for further analyses. Profiled analytes were excluded if i) detectable signal was absent in >33% of all global lipidomics study samples, or >4% of all targeted aqueous study samples, or ii) the coefficient of variation (CV) across all QC samples exceeded 20% (global) or 15% (targeted). 618 of 1706 lipid metabolite features had detectable signal intensities in ≧6796 of samples in both training and testing sets, and 372 of 618 were retained for analysis (CV≦20%). 98 of ˜160 metabolites in the targeted analysis had detectable signal intensity in ≧96% of all samples, and 90 of 98 were retained (CV≦15%). A total of four study samples were excluded entirely from either the global (n=2) or targeted (n=2) analysis due to loss of material during sample preparation. Targeted profiling measurements for an individual metabolite were normalized to the within-batch mean signal among QC samples for that metabolite. Global lipidomics measurements were not normalized, as all samples were run in one continuous batch.

Linear regression analyses were conducted in which log2-transformed metabolite values were regressed on case status (X, defined as 0/1), with adjustment for covariates (Cᵢ): M˜α₀+[α₁C₁+ . . . +αₙCₙ]+βX. Metabolites for which β differed significantly from zero (at P<0.05) in the training set were advanced to the testing set and similarly assessed, with those satisfying P<0.05 selected as preliminary candidates. Correction for multiple comparisons in the testing set was conducted using the Benjamini-Hochberg false discovery rate (FDR) approach, with Bonferroni-level significance also evaluated. Inclusion of an individual covariate (Cᵢ) in a given regression model was determined by assessment of the association between values of the metabolite and the covariate in control samples; M˜α+γᵢCᵢ|X=0. Covariates for which γᵢ differed significantly from zero (at P<0.05) were included in the primary regression model for the selected metabolite. Adjustment was considered for several OC risk factors—age (grouped-linear: 54-64, 65-74, 75+), BMI (grouped-linear: 18.5-24.9, 25-29.9, 30+), parity (grouped-linear; NP, 1, ≧2), oral contraceptive use (yes/no)—and duration of specimen storage (grouped-linear: 2-3, 4-5, or 6-8 y). Fold changes in the geometric means of metabolite signals between cases and controls were calculated using covariate-adjusted metabolite values. For a given metabolite, if covariates C₁ and C₂ were included in the main regression model, adjusted metabolite values were obtained as M_{adj}=M_{unadj}−δ₁C₁−δ₂C₂, where δ₁ and δ₂ are coefficients derived from the regression M−α+δ₁C₁+δ₂C₂|X=0.

### Classification Models

Multivariate classifiers for discriminating cases from controls were constructed using regularized logistic regression with an elastic net penalty (R package: glmnet) [35]. After exclusion of 14 subjects with missing CA125 values and two subjects missing all global lipidomics profiles, 84 subjects (44 cancers and 40 benign controls) were retained for analysis. Missing values in the lipidomics dataset were imputed using the k-nearest neighbor method (R package: impute), after exclusion of analytes with non-detectable signal in >33% of study samples. Cases and controls were randomly allocated to a training set (75%; n=63), used for variable selection and model selection, or a testing set (25%; n=21), used for (preliminary) model validation. With the mixing parameter (a) set to 0.5, five-fold cross-validation was conducted within the training set only to select the optimal value of the penalty parameter (λ). Using this value of λ, a model was generated using the complete training set data and used to predict class values for subjects in the testing set. For a hybrid model based on multiple metabolites and CA125, the coefficient penalty factor for CA125 was set to zero (versus one) to force inclusion of this predictor. Monte Carlo cross validation (MCCV) was conducted, such that the entire procedure described was repeated using 100 different training (and associated testing) sets randomly selected from the study sample. Mean estimated area under the receiver-operating curve (AUC) was calculated across the 100 testing sets. Performance of CA125 alone was estimated using the same MCCV framework. Composite average ROC curves were constructed to summarize overall classification accuracy (R package: ROCR) [36]. All univariate statistical analyses were conducted using Stata v13.1 (College Station, Tex.), while multivariate modeling was conducted using R v3.03, as indicated.

### Results

Demographic and reproductive characteristics for serous ovarian cancer cases and benign serous controls are shown in Table Cases and controls are well-balanced with respect to frequency-matching covariates: age, race, BMI, parity, and oral contraceptive use. None of the observed differences in the distributions of these variables between cases and controls, in either the training or testing set, reached statistical significance when assessed by X² tests (data not shown). Similarly, covariate distributions were highly similar between controls (or cases) in the training versus testing set. The majority of included cases (˜80%) were Stage III disease, with only 6% of cancers classified as Stage IV.

TABLE 1
Subject characteristics

| | Training set | | | | Testing set | | | |
|---|---|---|---|---|---|---|---|---|
| | Controls | | Cases | | Controls | | Cases | |
| | (n = 25) | | (n = 25) | | (n = 25) | | (n = 25) | |
| | n | % | n | % | n | % | n | % |
| Age | | | | | | | | |
| 54-64 | 10 | 40 | 16 | 64 | 10 | 40 | 16 | 64 |
| 65-74 | 9 | 36 | 5 | 20 | 9 | 36 | 6 | 24 |
| 75+ | 6 | 24 | 4 | 16 | 6 | 24 | 3 | 12 |
| Race/ethnicity^{a} | | | | | | | | |
| White | 19 | 95 | 21 | 95.5 | 19 | 100 | 21 | 95.5 |
| Non-white | 1 | 5 | 1 | 4.5 | 0 | 0 | 1 | 4.5 |
| BMI (kg/m²)^{a,b} | | | | | | | | |
| 18.5-24.9 | 7 | 36.8 | 7 | 33.3 | 5 | 29.4 | 7 | 33.3 |
| 25-29.9 | 4 | 21.1 | 5 | 23.8 | 6 | 35.3 | 10 | 47.6 |
| 30.0+ | 8 | 42.1 | 9 | 42.9 | 6 | 35.3 | 4 | 19 |
| Parity^{a} | | | | | | | | |
| NP^{c} | 2 | 11.1 | 3 | 15.8 | 2 | 10.5 | 3 | 14.3 |
| 1 | 2 | 11.1 | 4 | 21.1 | 3 | 15.8 | 3 | 14.3 |
| 2-3+ | 14 | 77.8 | 12 | 63.2 | 14 | 73.7 | 15 | 71.4 |
| OC use^{a,d} | | | | | | | | |
| No | 8 | 40 | 4 | 19 | 6 | 33.3 | 6 | 27.3 |
| Yes | 12 | 60 | 17 | 81 | 12 | 66.7 | 16 | 72.7 |
| Stage | | | | | | | | |
| I | | | 3 | 12 | | | 2 | 8 |
| II | | | 2 | 8 | | | 1 | 4 |
| III | | | 19 | 76 | | | 20 | 80 |
| IV | | | 1 | 4 | | | 2 | 8 |

^{a}Numbers may not add to total subjects due to missing data
^{b}Body mass index,
^{c}Nulliparous,
^{d}Oral contraceptive use

### Metabolite Profiling

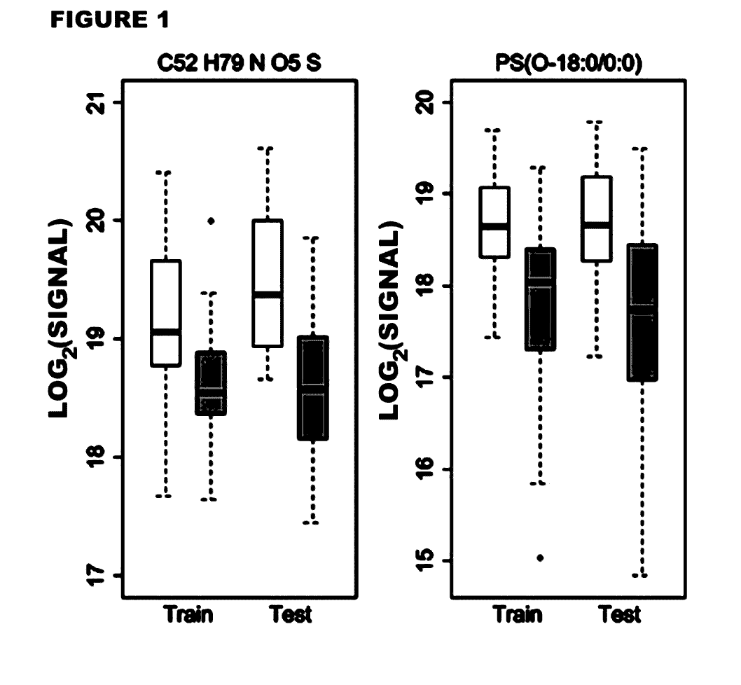

Global lipidomics profiling conducted using the LC-Q-TOF-MS system provided high sensitivity, resolution, and mass accuracy for a large number of lipids. 86 of 372 metabolites assessed exhibited a nominally significant (P<0.05) signal when comparing cases to controls in the training set, and 34 of these 86 reached significance (P<0.05) in the testing set. After correction for multiple comparisons, 17 metabolites remained significant (FDR q<0.05) (Table 2A), with two satisfying the highly stringent Bonferroni threshold (P<0.05/86=5.81×10⁻⁴) (FIG. 1). All 17 of these metabolites, which included a number of glycerolipids and glycerophospholipids, were found to be decreased in abundance in cases relative to controls, in both the training and testing sets. Fold changes in the testing set ranged from 0.43 to 0.67, with similar ratios observed in the training set.

Targeted aqueous profiling of the same 100 specimens was also conducted using an optimized LC-MS platform. Nine of 90 metabolites assessed exhibited differential abundance in cases versus controls in the training set (P<0.05), while only a single metabolite of these nine, the amino acid alanine, reached significance (P<0.05, q=0.22) in the testing set (Table 2B). Alanine showed reduced signal in cases relative to controls in both training and testing sets (fold change 0.83-0.86).

TABLE 2
Top metabolite marker candidates identified in global lipidomics and targeted aqueous profiling

| | Global Lipidomics | Training set (n = 49) | | | | | Testing set (n = 49) | | | | | |
|---|---|---|---|---|---|---|---|---|---|---|---|---|
| A | Metabolite | N^{a} | | FC^{b} | P^{c} | Cov^{d} | N^{a} | | FC^{b} | P^{c} | Cov^{d} | q^{e} |
| 1 | C52 H79 N O5 S | 42 | ▾ | 0.69 | 0.0056 | | 39 | ▾ | 0.56 | 0.0001^{#} | | 0.01 |
| 2 | PS(O-18:0/0:0) | 47 | ▾ | 0.56 | 0.0006 | | 46 | ▾ | 0.46 | 0.0004^{#} | | 0.02 |
| 3 | 18:3 Cholesteryl ester | 49 | ▾ | 0.75 | 0.0038 | | 49 | ▾ | 0.66 | 0.0010 | | 0.02 |
| 4 | TG(16:0/16:1(9Z)/16:1(9Z))[iso3] | 38 | ▾ | 0.55 | 0.0103 | 2; | 38 | ▾ | 0.43 | 0.0010 | 2; | 0.02 |
| 5 | PG(P-20:0/12:0) | 37 | ▾ | 0.73 | 0.0378 | | 35 | ▾ | 0.54 | 0.0010 | | 0.02 |
| 6 | TG(16:1(9Z)/16:1(9Z)/16:1(9Z)) | 39 | ▾ | 0.49 | 0.0021 | 2; | 38 | ▾ | 0.45 | 0.0012 | 2; | 0.02 |
| 7 | PS(O-18:0/16:1(9Z)) | 47 | ▾ | 0.56 | 0.0002 | | 48 | ▾ | 0.59 | 0.0015 | | 0.02 |
| 8 | TG(17:2(9Z,12Z)/17:2(9Z,12Z)/20:5 | | | | | | | | | | | |
| | (5Z,8Z,11Z,14Z,17Z))[iso3] | 45 | ▾ | 0.80 | 0.0440 | | 46 | ▾ | 0.67 | 0.0024 | | 0.02 |
| 9 | TG(16:1(9Z)/17:2(9Z,12Z)/ | | | | | | | | | | | |
| | 17:2(9Z,12Z))[iso3] | | | | | | | | | | | |
| 10 | TG(16:1(9Z)/17:1(9Z)/17:2(9Z,12Z)) | | | | | | | | | | | |
| | [iso6] | 39 | ▾ | 0.58 | 0.0037 | 2; 5 | 38 | ▾ | 0.52 | 0.0029 | 2; 5 | 0.02 |
| 11 | PE(18:1(9Z)/20:3(8Z,11Z,14Z)) | 44 | ▾ | 0.66 | 0.0054 | | 48 | ▾ | 0.61 | 0.0035 | | 0.03 |
| 12 | C57 H102 N2 O S | 38 | ▾ | 0.51 | 0.0116 | 2; | 37 | ▾ | 0.46 | 0.0040 | 2; | 0.03 |
| 13 | C29 H47 N9 O2 | 48 | ▾ | 0.48 | 0.0001 | | 49 | ▾ | 0.46 | 0.0044 | | 0.03 |
| 14 | TG(16:0/16:0/16:1(9Z))[iso3] | 37 | ▾ | 0.54 | 0.0345 | 2; | 37 | ▾ | 0.46 | 0.0044 | 2; | 0.03 |
| 15 | PS(O-20:0/0:0) | 46 | ▾ | 0.64 | 0.0082 | | 47 | ▾ | 0.60 | 0.0048 | | 0.03 |
| 16 | PE(18:1(9Z)/20:3(8Z,11Z,14Z)) | 48 | ▾ | 0.73 | 0.0226 | | 46 | ▾ | 0.65 | 0.0049 | | 0.03 |
| 17 | TG(16:1(9Z)/17:0/ | | | | | | | | | | | |
| | 17:2(9Z,12Z)) [iso6] | 39 | ▾ | 0.68 | 0.0221 | 2; | 38 | ▾ | 0.55 | 0.0052 | 2; | 0.03 |
| | Targeted Aqueous Profiling | Training set (n = 48) | | | | | Testing set (n = 50) | | | | | |
| B | Metabolite | N^{a} | | FC^{b} | P^{c} | Cov^{d} | N^{a} | | FC^{b} | P^{c} | Cov^{d} | q^{e} |
| 1 | Alanine | 48 | ▾ | 0.828 | 0.0416 | | 50 | ▾ | 0.862 | 0.0246 | | 0.221 |

^{a}Number of subjects with detectable signal for indicated metabolite and non-missing covariate values where applicable,
^{b}Fold change of geometric mean signal, comparing cases to controls,
^{c}P value derived from linear regression of metabolite values on case status, with adjustment for indicated covariates,
^{d}Covariates included in linear regression model: age (1), BMI (2), parity (3), OC use (4), specimen storage duration (5),
^{e}False discovery rate (Benjamini and Hochberg),
^{#}Significant after Bonferroni correction

### Multivariate Classification Models

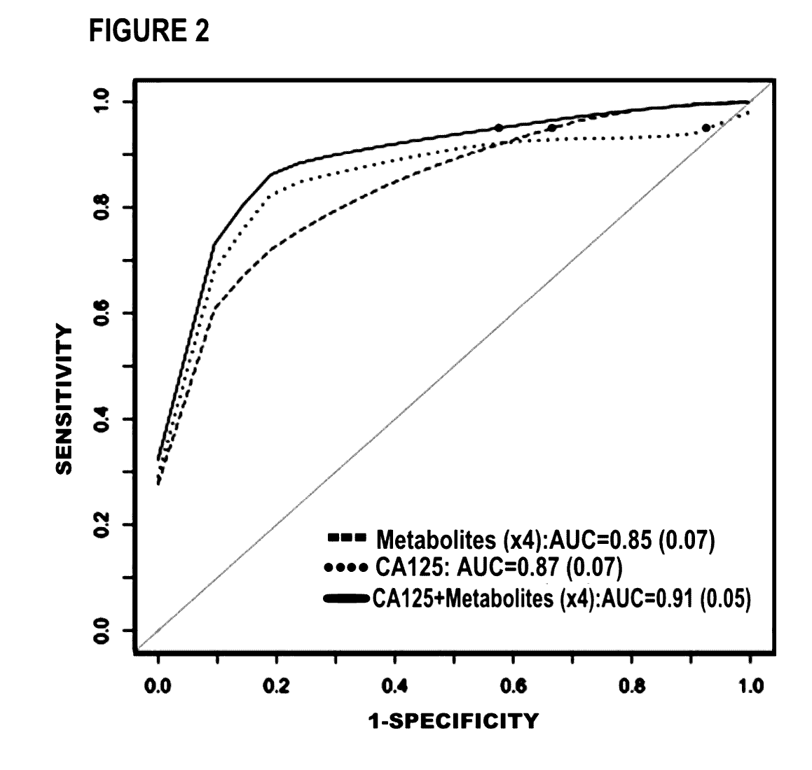

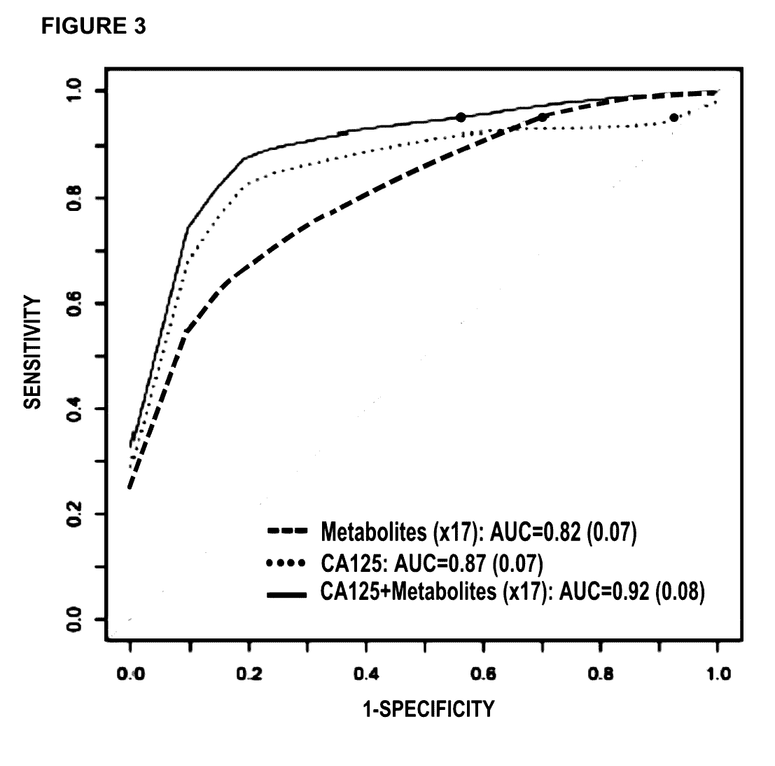

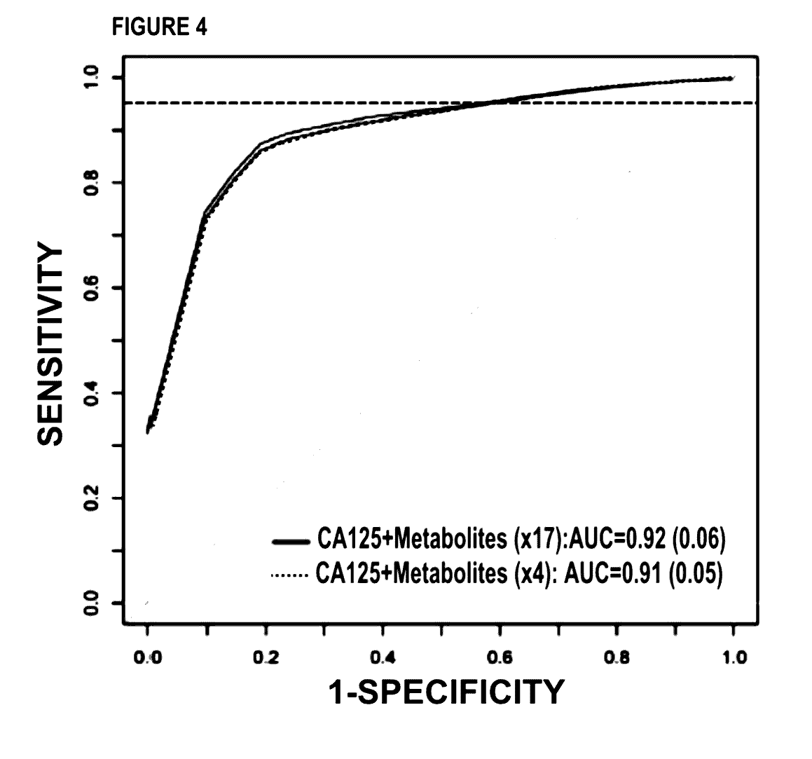

To explore the utility of combining multiple metabolites into a composite panel for discriminating between benign and malignant serous ovarian tumors, we selected the top four lipid metabolites listed in Table 2A, and conducted multivariate modeling, after imputing missing values via the k-nearest neighbor method. The mean AUC of models built using these four lipid markers, and assessed across 100 rounds of MCCV, was 0.85 (SD=0.07), with estimated specificity of ˜35% at 95% sensitivity (FIG. 2). Given the availability of CA125 plasma measurements on the majority of study participants, we also assessed the classification accuracy of a multi-marker model based on both CA125 and these four lipid metabolites, relative to CA125 alone. Performance assessments via MCCV were suggestive of a modest gain in overall classification accuracy for the hybrid model relative to CA125 alone (mean AUC 0.91 versus 0.87, respectively), but observed AUC differences did not reach statistical significance. At high fixed levels of sensitivity (eg. 95%), we observed substantially higher levels of estimated specificity for the joint model (˜43%) versus CA125 alone (<10%). Expanded models built using all 17 lipid metabolites listed in Table 2A (FDR q<0.05) achieved very similar accuracy (FIGS. 3-4)

### Discussion

Ovarian cancer is a leading cause of cancer-related mortality among U.S. women [1]. As most of these cancers are detected at late and incurable stages, an overarching long-term research objective is the development of novel non-invasive methods for early detection that could be implemented in population-based screening programs [37]. In parallel, a second important challenge, likely more tractable in the short-term, relates to the effective clinical triage of women presenting with a pelvic/ovarian mass, and specifically the ability to discriminate benign from malignant tumors [13]. New clinical tools based on blood-based protein markers have recently become available to address this second challenge [4, 5], but evaluation remains ongoing, and performance improvement remains a key goal. We employed two independent metabolomics profiling approaches and screened for plasma metabolite biomarkers that can differentiate benign from malignant serous ovarian tumors. Seventeen lipid metabolites were identified as differentially abundant (all reduced) in the plasma of cases versus controls. When combined into a multivariate classifier, four of these markers exhibited good discriminatory ability (AUC=0.85), and showed further potential in a hybrid model (AUC=0.91) to enhance the accuracy achieved by CA125 alone (AUC=0.87), especially at fixed high levels of sensitivity.

A number of metabolomics-based studies of ovarian cancer in the past decade have provided evidence of metabolite alterations in tissue, blood, and urine associated with the presence of OC; suggested the utility of metabolite-based classifiers for differentiating disease states; and offered initial insights into metabolic perturbations linked to this cancer [25-32, 38, 39]. This body of work has also highlighted a number of familiar challenges in the metabolomics field: small sample sizes; differences in study designs, inclusion criteria, case definitions, sample protocols, profiling platforms, and analysis approaches; and identification of many unknown “features”. These factors (among others) likely underlie the limited concordance observed among prior reports, with respect to specific metabolite markers and signatures. In the largest study to date [30], a total of 53 differential metabolites were identified in plasma from cases with epithelial OC versus benign ovarian tumor (BOT), including phospholipids, acylcarnitines, amino acid metabolites, and other analytes. Four metabolites described in a previous independent study [29] (by the same authors) were also identified in this larger analysis, including two lysophospholipids (LPC(18:2) and LPC(14:0)). Plasma concentrations of several phospholipids were reduced in cases with Stage III/IV disease relative to BOT, but increased in early-stage cancers. Disturbances in lipid metabolism have also been suggested by at least two other metabolomics reports [25, 40]. None of these studies deeply interrogated the tissue or plasma lipidome, however, as standard widely-used extraction procedures and platforms were employed.

Of the 35 differentially-abundant metabolite (P<0.05) identified in the primary analyses, the vast majority (34/35) were identified via lipidomics, with only a single metabolite (alanine) found using our aqueous platform. The 34 lipid metabolites included 16 glycerophospholipids, eight glycerolipids (all triacylglycerols), one sphingolipid, one sterol lipid, and eight unknowns, while the top 17 candidates (q<0.05) comprised seven glycerolipids, six glycerophospholipids, one sterol lipid, and three unknowns. Strikingly, the plasma levels of all 34 of these lipids were reduced in cases versus benign controls. While altered lipid metabolism has been linked to OC by past studies, specific findings have been inconsistent, with either increases or decreases in various phospholipids and lipid precursors reported in both tissue and plasma from OC cases [25, 26, 30, 40, 41]. Lipid alterations in the context of OC pathogenesis may reflect higher rates of cell division [25], enhanced activity of phospholipase A2 and inflammation [30], elevated PI3-kinase activity, and increased membrane degradation or changes in cellular morphology [40]. However, the extent to which circulating plasma lipid metabolite levels correlate with those found in ovarian tissue remains uncertain. Future parallel analyses of paired pre-operative plasma and resected tissue specimens isolated from the same subjects may help clarify this relationship.

Clinical discrimination of benign from malignant ovarian tumors remains an important challenge with significant implications for patient prognosis and survival. Women with OC who are referred to tertiary care specialists have increased survival relative to those treated by general gynecologists/surgeons, likely reflecting optimal surgical debulking and clinical management [2, 3] Treatment of women with benign tumors by specialists does not appear to confer medical benefit (or harm), but increases resource utilization and may elevate patient anxiety. A cost-benefit simulation analysis [42] has argued for universal referral of women with an ovarian mass to specialized tertiary care, and suggested that any triaging plan should prioritize high sensitivity, while secondarily maximizing specificity. Referral has typically been based on a combination of personal history, clinical examination, ultrasound, and CA125 blood levels, components of which were formalized into an algorithm in 1990 known as the Risk of Malignancy Index (RMI) [43], Two additional algorithms have been developed; ROMA, based on serum CA125 and HE4 with menopausal status [4]; and OVA1, based on CA125 in combination with additional blood protein markers discovered via mass spectrometry [5-8]. The only available head-to-head evaluation of these two approaches was a recent small study (50 post-menopausal benign controls and 26 post-menopausal ovarian cancers), which reported 42% specificity at 96% sensitivity for OVA1, and 76% specificity at 88.5% sensitivity for ROMA [9]. This level of performance for OVA1 appears quite similar to that of our hybrid model incorporating CA125 and multiple lipid metabolites.

The present study represents the first global lipidomics profiling analysis of OC, and is atypical of past reports in that it integrates data from two independent metabolomics platforms. Together, the profiling approaches employed provided broad coverage of both small lipids and aqueous analytes. Global lipid profiling enabled far more comprehensive interrogation of this important class of metabolites than has been reported in >10 past metabolomics studies of OC. We restricted our analysis to the subset of metabolites that exhibited the most reproducible profiles based on assessment of included QC samples (CV≦20%), thereby focusing attention on the most robust signals. Our use of a training-testing set framework with adjustment for multiple comparisons allowed for rigorous statistical assessment and minimization of type I error, while inclusion of several covariates in our regression models helped control for the effects of potential confounders, and increased the probability of identifying metabolite markers independent of known risk factors. The use of penalized regression methods further allowed for the development of optimized classifiers based on inclusion of an expanded pool of predictors variables.

This work also has certain limitations. First, the overall sample size of 50 cases and 50 controls restricted the statistical power and likely resulted in a number of missed candidate markers among the profiled metabolites. The extent of missing data (˜20%) for several covariates such as BMI, parity, and oral contraceptive use further reduced the number of available samples for our primary regression analyses, while missing values for CA125 (˜14%) limited the pool of participants in which we could assess hybrid CA125-metabolite classification models. Second, a number of “unknown features” were identified in the global analysis, as the corresponding MS peaks could not be mapped to compounds in the Agilent and METLIN chemical libraries. Third, the study design was retrospective, and the extent to which selected participants with serous OC or serous BOT were representative of the larger populations of women with these conditions remains unknown. While serous OC and BOT comprise a large percentage of ovarian masses, real-world prospective triaging also requires discrimination between benign and malignant tumors of multiple disparate histologies. With respect to the multivariate modeling, it should be noted that our estimates of classification accuracy were obtained using the same samples used to identify the included metabolite predictors; thus external validation will be essential to obtain truly unbiased performance estimates. Lastly, the cases included in the present study sample consisted primarily of women with Stage III tumors, likely reflecting the stage distribution most commonly seen among OC patients presenting with an ovarian mass. It remains to be determined whether the identified candidate markers also exhibit similar fluctuations at earlier phases of disease progression. Of interest, recent evidence from prospective multi-institutional trials supports the potential utility of the OVA1 test for identifying early-stage OC [44].

The results described herein provide evidence that alterations in circulating plasma lipid metabolites are associated with the presence of malignant ovarian carcinoma (versus benign ovarian tumor). The use of a global profiling platform focused specifically on lipid molecules has significantly expanded the pool of candidate markers that may be implicated in, or reflective of, the OC disease process. Additional studies in larger independent populations are required for validation. It will be of significant interest to determine if combining plasma metabolites with established protein-based markers (eg. CA125, HE4, prealbumin, transferrin) may enable superior classification of benign versus malignant tumors, and ultimately lead to the more effective clinical triage of women presenting with an ovarian mass.

### References

1. Siegel R L, Miller K D, Jemal A (2015) Cancer Statistics, 2015. CA Cancer J Olin 65:5-29, doi: 10.3322/caac.21254.
2. Bristow R E, Tomacruz R S, Armstrong D K, et al. (2002) Survival effect of maximal cytoreductive surgery for advanced ovarian carcinoma during the platinum era: a meta-analysis. J Clin Oncol 20:1248-59.
3. Engelen M J a, Kos H E, Willemse P H B, et al. (2006) Surgery by consultant gynecologic oncologists improves survival in patients with ovarian carcinoma. Cancer 106:589-98. doi: 10.1002/cncr.21616
4. Moore R G, McMeekin D S, Brown A K, et al. (2009) A novel multiple marker bioassay utilizing HE4 and CA125 for the prediction of ovarian cancer in patients with a pelvic mass. Gynecol Oncol 112:40-46, doi: 10.1016/j.ygyno,2008.08.031
5. Ueland F R, Desimone C P, Seamon L G, et al. (2011) Effectiveness of a multivariate index assay in the preoperative assessment of ovarian tumors. Obstet Gynecol 117:1289-1297. doi: 10.1097/AOG.0b013e31821b5118
6. Kozak K R, Amneus M W, Pusey S M, et al. (2003) Identification of biomarkers for ovarian cancer using strong anion-exchange ProteinChips: potential use in diagnosis and prognosis. Proc Natl Acad Sci U S A 100:12343-8. doi: 10.1073/pnas.2033602100
7. Kozak K R, Su F. Whitelegge J P, et al. (2005) Characterization of serum biomarkers for detection of early stage ovarian cancer. Proteomics 5:4589-96. doi: 10.1002/pmic.200500093
8. Su F, Lang J, Kumar A, et al. (2007) Validation of candidate serum ovarian cancer biomarkers for early detection. Biomark Insights 2:369-75.
9. Grenache D G, Heichman K a, Werner T L, Vucetic Z (2015) Clinical performance of two multi-marker blood tests for predicting malignancy in women with an adnexal mass. Olin Chim Acta 438:358-63. doi: 10.1016/j.cca.2014.09.028
10. Bristow R E, Smith A, Zhang Z, et al. (2013) Ovarian malignancy risk stratification of the adnexal mass using a multivariate index assay. Gynecol Oncol 128:252-259. doi: 10.1016/j.ygyno.2012.11.022
11. Moore R G, Miller M C, Disilvestro P, et al. (2011) Evaluation of the diagnostic accuracy of the risk of ovarian malignancy algorithm in women with a pelvic mass. Obstet Gynecol 118:280-288, doi: 10.1097/AOG.0b013e318224fce2
12. Sandri M T, Bottari F, Franchi D, et al. (2013) Comparison of HE4, CA125 and ROMA algorithm in women with a pelvic mass: Correlation with pathological outcome. Gynecol Oncol 128:233-238. doi: 10.1016/j.ygyno.2012.11.026
13. Nolen B M, Lokshin A E (2013) Biomarker testing for ovarian cancer: clinical utility of multiplex assays, Mol Diagn Ther 17:139-46, doi: 10.1007/s40291-013-0027-6
14. Bast R C, Skates S, Lokshin A, Moore R G (2012) Differential diagnosis of a pelvic mass: improved algorithms and novel biomarkers. Int J Gynecol Cancer 22 Suppl 1:S5-8. doi: 10,1097/IGC.0b013e318251c97d
15. O'Connell T M (2012) Recent advances in metabolomics in oncology. Bioanalysis 4:431-451, doi: 10.4155/bio.11.326
16. Gu H. Gowda G A N, Raftery D (2012) Metabolic profiling: are we en route to better diagnostic tests for cancer? Future Oncol 8:1207-10. doi: 10.2217/fon.12.113
17. Liesenfeld D B, Habermann N, Owen R W, et al. (2013) Review of mass spectrometry-based metabolomics in cancer research. Cancer Epidemiol Biomarkers Prev 22:2182-201. doi: 10.1158/1055-9965.EPI-13-0584
18. Nicholson J K, Lindon J C, Holmes E (1999) {‘Metabolomics’:} understanding the metabolic responses of living systems to pathophysiological stimuli via multivariate statistical analysis of biological {NMR} spectroscopic data. Xenobiotica 29:1181-1189. doi: 10.1080/004982599238047
19. Gowda G A, Zhang S, Gu H, et al. (2008) Metabolomics-based methods for early disease diagnostics. Expert Rev Mol Diagn 8:617-633. doi: 10,1586/14737159.8.5.617
20. Sreekumar A, Poisson L M, Rajendiran T M, et al. (2009) Metabolomic profiles delineate potential role for sarcosine in prostate cancer progression. Nature 457:910-914. doi: nature07762 [pii] 10.1038/nature07762
21. Asiago V M, Alvarado L Z, Shanaiah N, et al. (2010) Early Detection of Recurrent Breast Cancer Using Metabolite Profiling. Cancer Res 70:8309-8318. doi: 10.1158/0008-5472.CAN-10-1319
22. Wei S, Suryani Y, Gowda G a N, et al. (2012) Differentiating hepatocellular carcinoma from hepatitis C using metabolite profiling. Metabolites 2:701-16. doi: 10.3390/metabo2040701
23. Zhang J, Bowers J, Liu L, et al. (2012) Esophageal cancer metabolite biomarkers detected by LC-MS and NMR methods. PLoS One 7:e30181. doi: 10.1371/journal.pone.0030181
24. Zhu J, Djukovic D, Deng L, et al. (2014) Colorectal Cancer Detection Using Targeted Serum Metabolic Profiling.
25. Denkert C, Budczies J, Kind T, et al. (2006) Mass spectrometry-based metabolic profiling reveals different metabolite patterns in invasive ovarian carcinomas and ovarian borderline tumors. Cancer Res 66:10795-10804. doi: 10.1158/0008-5472.CAN-06-0755
26. Fong M Y, McDunn J, Kakar S S (2011) Identification of metabolites in the normal ovary and their transformation in primary and metastatic ovarian cancer. PLoS One 6:1-12. doi: 10.1371/journal.pone.0019963
27. Odunsi K, Wollman R M, Ambrosone C B, et al. (2005) Detection of epithelial ovarian cancer using 1H-NMR-based metabonomics. Int J Cancer 113:782-788. doi: 10.1002/ijc.20651
28. Zhou M, Guan W, Walker L D, et al. (2010) Rapid mass spectrometric metabolic profiling of blood sera detects ovarian cancer with high accuracy. Cancer Epidemiol Biomarkers Prev 19:2262-2271. doi: 10.1158/1055-9965.EPI-10-0126
29. Zhang T, Wu X, Yin M, et al. (2012) Discrimination between malignant and benign ovarian tumors by plasma metabolomic profiling using ultra performance liquid chromatography/mass spectrometry. Clin Chim Acta 413:861-868. doi: 10.1016/j.cca.2012.01.026
30. Ke C, Hou Y, Zhang H, et al. (2014) Large-scale profiling of metabolic dysregulation in ovarian cancer. Int J Cancer 00:1-11. doi: 10.1002/ijc.29010
31. Slupsky C M, Steed H, Wells T H, et al, (2010) Urine metabolite analysis offers potential early diagnosis of ovarian and breast cancers. Clin Cancer Res 16:5835-5841, doi: 10.1158/1078-0432.CCR-10-1434
32. Zhang T, Wu X, Ke C, et al, (2013) Identification of potential biomarkers for ovarian cancer by urinary metabolomic profiling. J Proteome Res 12:505-12. doi: 10.1021/pr3009572
33. Palmer C, Duan X, Hawley S, et al. (2008) Systematic evaluation of candidate blood markers for detecting ovarian cancer. {PLoS} One 3:e2633. doi: 10.1371/journal.pone.0002633
34. Scholler N, Crawford M, Sato A, et al. (2006) Bead-based ELISA for validation of ovarian cancer early detection markers. Clin Cancer Res 12:2117-2124. doi: 10.1158/1078-0432.CCR-05-2007
35. Friedman J, Hastie T, Tibshirani R (2010) Regularization Paths for Generalized Linear Models via Coordinate Descent. J Stat Softw 33:1-22.
36. Sing T, Sander O, Beerenwinkel N, Lengauer T (2005) ROCR: visualizing classifier performance in R. Bioinformatics 21:3940-1. doi: 10.10931bioinformatics/bti623
37. Cohen J G (2014) In 2014, can we do better than CA125 in the early detection of ovarian cancer? World J Biol Chem 5:286. doi: 10.4331/wjbc.v5.i3.286
38. Chen J, Zhang X, Cao R, et al, (2011) Serum 27-nor-5β-cholestane-3,7,12,24,25 pentol glucuronide discovered by metabolomics as potential diagnostic biomarker for epithelium ovarian cancer. J Proteome Res 10:2625-2632, doi: 10.1021/pr200173q
39. Garcia E, Andrews C, Hua J, et al. (2011) Diagnosis of early stage ovarian cancer by {1H} {NMR} metabonomics of serum explored by use of a microflow {NMR} probe. J Proteome Res 10:1765-1771. doi: 10.1021/pr101050d
40. Ferna F M (2014) Metabolomic Serum Profiling Detects Early-Stage High-Grade Serous Ovarian Cancer in a Mouse Model: doi: 10.1021/pr5009948
41. Sutphen R, Xu Y, Wilbanks G D, et al. (2004) Lysophospholipids are potential biomarkers of ovarian cancer. Cancer Epidemiol Biomarkers Prev 13:1185-1191. doi: 13/7/1185 [pii]
42. Havrilesky Dinan M, Sfakianos G P, et al. (2015) Costs, Effectiveness, and Workload Impact of Management Strategies for Women With an Adnexal Mass. 1-10. doi: 10.1093/jnci/dju322
43. Jacobs I, Oram D, Fairbanks J, et al. (1990) A risk of malignancy index incorporating CA 125, ultrasound and menopausal status for the accurate preoperative diagnosis of ovarian cancer. Br J Obstet Gynaecol 97:922-9.
44. Longoria T C, Ueland F R, Zhang Z, et al. (2014) Clinical performance of a multivariate index assay for detecting early-stage ovarian cancer. Am J Obstet Gynecol 210:78.e1-9. doi: 10.1016/j.ajog.2013.09.017.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention, Those skilled in the art will also appreciate that such equivalent embodiments do not depart from the spirit and scope of the invention as set forth in the appended claims.

What is claimed is:

1. A method of determining the amounts of metabolites in a sample, the method comprising:
(a) measuring the concentrations of at least two components of a panel of a plurality of metabolites in a sample obtained from a subject, wherein the components of the panel are selected from the group consisting of: C52 H79 N O5 S, PS(O-18:0/0:0), 18:3 Cholesteryl ester+22.7, TG(16:0/16:1(9Z)/16:1(9Z))[iso3]+23.5, PG(P-20:0/12:0), TG(16:1(9Z)/16:1(9Z)/16:1(9Z)) +22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z)[iso6], and Alanine; and
(b) determining a ratio of the concentration of each of the components measured in step (a) to control concentration of each of the components.

2. The method of claim 1, wherein the sample is plasma obtained from a subject suspected of having ovarian cancer (OC) and/or a subject having a pelvic tumor.

3. The method of claim 1, further comprising:
(c) detecting OC in the subject when the ratio determined in (b) is less than 1 for each of the components.

4. The method of claim 1, wherein the at least two components comprises Alanine.

5. The method of claim 1, wherein the at least two components comprises C52 H79 N O5 S and PS(O-18:0/0:0).

6. The method of claim 1, wherein the measuring comprises measuring the concentrations of at least 3 components of the panel.

7. The method of claim 6, wherein the at least 3 components of the panel comprises C52 H79 N O5 S, PS(O-18:0/0:0), and Alanine.

8. The method of claim 1, wherein the measuring comprises measuring the concentrations of at least 4 components of the panel.

9. The method of claim 8, wherein the at least 4 components of the panel comprises C52 H79 N O5 S, PS(O-18:0/0:0), 18:3 Cholesteryl ester+22.7, and TG(16:0/16:1(9Z)/16:1(9Z))[iso3]+23.5.

10. The method of claim 1, wherein the measuring comprises measuring the concentrations of at least 5 components of the panel.

11. The method of claim 10, wherein the at least 5 components of the panel comprises C52 H79 N O5 S, PS(O-18:0/0:0), 18:3 Cholesteryl ester+22.7, TG(16:0/16:1(9Z)/16:1(9Z))[iso3]+23.5, and Alanine.

12. The method of claim 1, wherein the measuring comprises measuring the concentrations of at least 17 components of the panel.

13. The method of claim 12, wherein the at least 17 components of the panel comprise: C52 H79 N O5 S, PS(-18:0/0:0), 18:3 Cholesteryl ester+22.7, TG(16:0/16:1(9Z)/16:1(9Z))[iso3]+23.5, PG(P-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), and TG(16:1(9Z)/17:0/17:2(9Z,12Z)[iso6].

14. The method of claim 1, comprising measuring the concentrations of at least 18 components of a panel of a plurality of metabolites in a sample from the subject, wherein the at least 18 components of the panel comprises C52 H79 N 05 S, PS(0-18:0/0:0), 18:3 Cholesteryl ester+22.7, TG(16:0/16:1(9Z)/16:1(9Z))[iso3]+23.5, PG(P-20:0/12:0), TG(16:1(9Z)/16:1(9Z)/16:1(9Z)) +22.7, PS(O-18:0/16:1(92)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z)[iso6], and Alanine.

15. The method of claim 1, further comprising measuring CA125 and/or HE4 in a sample from the subject, wherein a statistically significant increase in CA125 and/or HE4 relative to a control sample is indicative of OC.

16. The method of claim 1, further comprising measuring prealbumin and/or transferrin in a sample from the subject, wherein a statistically significant decrease in prealbumin and/or transferrin relative to a control sample is indicative of OC.

17. The method of claim 1, wherein the control sample is obtained from a normal, healthy subject or obtained from the subject at an earlier time.

18. The method of claim 1, wherein the measuring comprises liquid chromatography, mass spectrometry, enzymatic assay, and/or immunoassay.

19. A kit comprising a set of standards, wherein the set comprises at least three standards of the following: C52 H79 N O5 S, PS(O-18:0/0:0), 18:3 Cholesteryl ester +22.7, TG(16:0/16:1(9Z)/16:1(9Z))[iso3]+23.5, PG(P-20:0/12:0), TG(16:1(9Z)/16:1(9Z)/16:1(9Z))+22.7, PS(O-18:0/16:1(9Z)), TG(17:2(9Z,12Z)/17:2(9Z,12Z)/20:5(5Z,8Z,11Z,14Z,17Z))[iso3], TG(16:1(9Z)/17:2(9Z,12Z)/17:2(9Z,12Z))[iso3]+22.2, TG(16:1(9Z)/17:1(9Z)/17:2(9Z,12Z))[iso6], PE(18:1(9Z)/20:3(8Z,11Z,14Z)), C57 H102 N2 O S, TG(16:0/16:0/16:1(9Z))[iso3], C29 H47 N9 O2, PS(O-20:0/0:0), PE(18:1(9Z)/20:3(8Z,11Z,14Z)), TG(16:1(9Z)/17:0/17:2(9Z,12Z)[iso6].

20. The kit of claim 19, further comprising reagents for detection of alanine.
